(19) United States
(12) Patent Application Publication
(10) Pub. No.: US 2014/0336461 A1
(43) Pub. Date: Nov. 13, 2014

(54) SURGICAL STRUCTURED LIGHT SYSTEM

(71) Applicant: The Trustees of Columbia University in the City of New York, New York, NY (US)

(72) Inventors: Austin Reiter, Great Neck, NY (US); Peter K. Allen, Pleasantville, NY (US)

(21) Appl. No.: 14/341,500

(22) Filed: Jul. 25, 2014

Related U.S. Application Data
(63) Continuation of application No. PCT/US2013/038161, filed on Apr. 25, 2013.
(60) Provisional application No. 61/880,612, filed on Sep. 20, 2013.
(60) Provisional application No. 61/859,007, filed on Jul. 26, 2013.
(60) Provisional application No. 61/638,466, filed on Apr. 25, 2012.

(51) Int. Cl. A61B 1/00; A61B 1/313; A61B 1/06; A61B 1/05

(52) U.S. Cl. CPC A61B 1/00193; A61B 1/05; A61B 1/3132; A61B 1/06; A61B 1/00009
USPC 600/111

(57) ABSTRACT

A Surgical Structured Light (SSL) system is disclosed that provides real-time, dynamic 3D visual information of the surgical environment, allowing registration of pre- and intra-operative imaging, online metric measurements of tissue, and improved navigation and safety within the surgical field.

### RELATED APPLICATIONS

This application hereby claims priority to U.S. Provisional Patent Application No. 61/880,612 filed Sep. 20, 2013, and U.S. Provisional Patent Application No. 61/859,007, filed Jul. 26, 2013, and is a Continuation of International Application No. PCT/US13/38161, filed Apr. 25, 2013, which claims priority to U.S. Provisional Application No. 61/638,466 filed Apr. 25, 2012.

### BACKGROUND OF THE DISCLOSED SUBJECT MATTER

1. Field of the Disclosed Subject Matter

The disclosed subject matter relates to improved 3D imaging. Particularly, the present subject matter described here is a Surgical Structured Light (SSL) system that includes a real-time 3D sensor that measures and models the surgical site during a procedure. The present subject matter provides real-time, dynamic 3D visual information of the surgical environment using a standard laparoscopic setup. This capability allows registration of pre- and intra-operative imaging, online metric measurements of tissue, and improved navigation and safety within the surgical field.

The systems and techniques disclosed herein are suitable for a myriad of applications and embodiments wherever depth perception can be a critical parameter such as minimally invasive surgeries, forensic applications, military applications, and law enforcement.

2. Description of Related Art

Standard laparoscopes provide a 2D image of a surgical site, leaving the surgeon without depth perception and making surgery more difficult. Although current laparoscope technology can provide perceptual depth information, whereby the surgeon can view stereo cameras and the human brain fuses the images to perceive depth, currently no methods exist to computationally provide this information for the purposes of measurements and automation.

Surgeons routinely use a telescope and camera system to provide an image of the surgical site during minimally invasive surgery in the abdomen and in the chest. This delivers a crisp, high-resolution 2D image, but the lack of depth perception imposes the following limitations and constraints on the surgical team: limits and slows the surgeon's technical performance; fails to inform the surgeon about the spatial relationships of various organs and their components (i.e., blood vessels, ureters, bronchial tubes, etc.); prevents registration of intra-operative real-time images with pre-operative images; inadequate 3D geometric visualization with which to do intra-operative planning; prevents accurate intra-operative measurements; and creates a very long learning curve for surgeons.

Since 1990, surgeons have developed minimally invasive surgery (MIS) techniques by using an endoscope (laparoscope or thoracoscope) to visualize the surgical site in the abdomen or chest. These scopes, when combined with a high resolution camera, provide a crisp, bright image of the surgical site and eliminate the need for a large incision through which to view and complete the operation. However, this technology delivers a 2-dimensional (2D) image of a 3-dimension (3D) surgical site, and, because of the insertion site for the scope, the angle of view of the surgical site is severely limited. Although the surgeon can sometimes complete MIS with this technology, the loss of depth perception and ability to view the surgical site from multiple angles slows the surgeon, lengthens the learning curve, and, in some complex cases, prevents the surgeon from completing the operation without making a large incision.

The benefits of minimally invasive surgery are well established. Despite that, many surgeons have not learned to use these techniques, in large part because of the long learning curve associated with a 2D image. Technology that would routinely deliver 3D information to surgeons would not only increase the availability of minimally invasive surgery to more patients by shortening the learning curve, it would also lead to better surgical procedures.

Minimally invasive surgeries, such as laparoscopy and arthroscopy, have become standard of care surgical techniques because they require smaller incisions compared to traditional open surgery. Central to these surgical techniques are endoscopes with video cameras that allow surgeons to visualize interior body structures, as requisite for performing surgery.

The most common laparoscopic procedure in the United States is the cholecystectomy, which is the surgical removal of the gall bladder. Overall, the efficacy of laparoscopy varies slightly with the type of surgical procedure; cholecystectomy is typically the standard against which other procedures are judged. Recently, laparoscopic surgery has been proven to reduce the risk of hospital-acquired infections, which also shows that the shift from open surgery to minimally invasive methods is critically important for patients in the long term.

The laparoscopic surgery market focuses on the instrumentation used in the surgical procedure. Laparoscopic surgical products consist of three broad segments: visualization, access, and resection instruments. Visualization consists of laparoscopes and cameras, access will include trocars, suction/irrigation, and insufflation systems, and resection will include scissors or forceps and direct energy tools such as ultrasonic and electrocautery scalpels and vessel sealers. The visualization segment of laparoscopic surgery products consists of laparoscopes and surgical cameras. Laparoscopes consist of a telescopic rod-lens system, a fiber optic cable, and a light source.

In laparoscopic surgery, the components that are required at first to perform the procedure are the visualization instruments. A visualization instrument consists of the telescopic rod lens, a video camera, fiber optic cables, and a cold light source. Typically, the rod lens, fiber optic cable, and light source are combined in an integral medical instrument collectively known as the laparoscope, while the video camera remains a distinct, but inseparable element of the visualization system.

There is strong evidence that the outcomes of MIS are better than traditional open surgery for most abdominal and thoracic operations: reduced complications, much quicker recovery, and equal or better treatment of the underlying condition. The unmet need is an imaging system that would provide additional information with which the surgeon could more efficiently and successfully complete surgery using MIS techniques.

Current visualization techniques are wanting in their ability to provide depth information that can be crucial to surgeons. There is an urgent need for a system that allows surgeons to visualize internal body structured in three dimensions for more accurate and safe surgical procedures.

The presently disclosed subject matter meets the need for 3D imaging for minimally invasive complex intra-abdominal and intra-thoracic operations and significantly improve the experience of the surgeon and improves outcomes for patients.

SUMMARY OF THE DISCLOSED SUBJECT MATTER

The present subject matter described here is a Surgical Structured Light (SSL) system that includes a real-time 3D sensor that measures and models the surgical site during a procedure. The present subject matter provides real-time, dynamic 3D visual information of the surgical environment using a standard laparoscopic setup. This capability allows registration of pre- and intra-operative imaging, online metric measurements of tissue, and improved navigation and safety within the surgical field. By adding this 3D component, minimally-invasive surgery can be easier to learn and more widely used.

The presently disclosed subject matter provides a new imaging technology that provides 1) real-time 3D reconstructions of the surgery site, 2) unique, novel views not previously available using endoscopic imaging, and 3) metrology of various dimensions inside of a surgical site. The current gold standard for imaging during MIS provides only a 2D image by using a high definition (HD) video camera and a rod lens rigid endoscope.

The present disclosure alleviates the burden of conventional imaging systems which require the surgeon to view the 3D image in a console set away from the patient. The present subject matter uses modern computation capabilities to generate an image with which the surgeon can precisely measure the size of or distance between structures in the surgical field, visualize the relationship between various structures, and register the intra-operative image with preoperative images. These additional data points should improve the efficiency, safety, and overall quality of MIS.

Laparoscopy is a minimally invasive surgical technique used in a wide range of abdomen operations. The surgeon performs the procedure under the skin with the assistance of a camera system. Standard laparoscopes utilize 2D imaging techniques, which limit depth perception and can restrict or slow surgeon performance, create a long learning curve for new surgeons, and create problems during surgery. This technology gives the standard laparoscope real-time, dynamic 3D imaging capabilities of the surgical scene for routine and complex laparoscopic procedures. The added dimension can significantly improve surgeon experience and potentially improve outcomes for patients.

Further, by minimizing depth perception issues, 3D imaging can provide shorter surgery times and improved navigation and safety, thus promoting wider use of minimally invasive surgery.

In an exemplary embodiment, the imaging system comprises a first camera; a second camera; a light source, the light source producing light at a frequency invisible to a human eye; a dispersion unit, the dispersion unit projecting a predetermined pattern of light from the invisible light source; an instrument, the instrument projecting the predetermined pattern of invisible light onto a target area; a band pass filter, the band pass filter directing visible light to the first camera and the predetermined pattern of invisible light to the second camera; wherein the second camera images the target area and predetermined pattern of invisible light, and computes a three-dimensional image.

Additionally, the first camera displays a visible image to an operator, and the light source produces a continuous ray of infrared light. In some embodiments, the three-dimensional image is computed from a single image of the target area. Advantageously, the second camera can be configured to take real time intra-operative images of the target area, and compare pre-operative images with intra-operative images.

In some embodiments, a RGB color is assigned to select locations of the second camera image, and a processor measures at least one length (e.g., depth) of the target area. In some instances the instrument is a laparoscope which includes a beam splitter disposed at an end thereof The laparoscope can include first and second optical channels, wherein the first optical channel projects the predetermined pattern of light onto the target area, and the second optical channel receives all available light from the target area.

In accordance with another aspect of the disclosed subject matter, a method for creating a three dimensional image comprises providing a first camera; providing a second camera; generating a ray of light at a frequency invisible to a human eye; dispersing the ray of invisible light through a dispersion unit; projecting a predetermined pattern of invisible light onto a target area; receiving the predetermined pattern of invisible light and visible light from the target area; directing visible light to the first camera and the predetermined pattern of invisible light to the second camera; imaging the target area and predetermined pattern of invisible light; and computing a three-dimensional measurement.

In some embodiments, the ray of invisible light is infrared light and the three-dimensional measurement is computed from a single image of the target area. Additionally, the second camera can be configured to capture real time intra-operative images of the target area, and a processor measures at least one length of the target area.

In another exemplary embodiment, an imaging system comprising a first camera; a second camera; a light source, the light source producing light at a frequency visible to a human eye; a dispersion unit, the dispersion unit projecting a predetermined pattern of light of a known range of frequencies from the light source; an instrument, the instrument projecting the predetermined pattern of light of a known range of frequencies onto a target area; a beam splitter, the beam splitter directing a first ray of light towards the first camera and a second ray of light towards the second camera; a notch filter, the notch filter filtering the second ray of light to the predetermined pattern of light of a known range of frequencies; wherein the second camera images the target area and predetermined pattern of light to compute a three-dimensional image.

### BRIEF DESCRIPTION OF THE DRAWINGS

A detailed description of various aspects, features, and embodiments of the subject matter described herein is provided with reference to the accompanying drawing, which is briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 provides a schematic view of one embodiment of the disclosed subject matter.

FIG. 2 is an exemplary embodiment of a Surgical Structured Light (SSL) system in accordance with the disclosed subject matter.

FIG. 3 is an exemplary embodiment of a customized dispersion mask in having a template or pattern, against which all other pattern images are matched in order to reconstruct in 3D. The image is taken at a flat plane, at a known distance, which is parallel to the distal tips of the side-by-side laparoscopes.

FIG. 4A is a representative image obtained by the SSL system disclosed herein depicting a plastic organ model of a stomach imaged using the white light imaging camera. (None of the blue light from the LED pattern projector is present in this image, which is presented to the surgeon as the main 2D imaging source during a procedure.

FIG. 4B is a representative projected mask pattern with the blue light on the same stomach model, used to reconstruct the object densely in 3D.

FIG. 5 is an exemplary embodiment of a hand held device in accordance with the disclosed subject matter. In this embodiment a 2-axis button joystick is provided as to allow the surgeon to perform virtual transformations (translate, rotate and scale) to 3D reconstructed images or Zoom and Pan 2D Images without having to take her/his hands of the laparoscopic tools or change the position of the laparoscopic camera.

FIG. 6 depicts a representative screen capture of our user interface. Left: Picture of a 2D Image of the cylinder object. Right: The 3D reconstructed model of the same cylinder using the SSL system. Both the 2D and 3D image information can be displayed to the surgeon in a useable and intuitive fashion. Because colorized 3D point cloud information is provided, the surgeon is able to move, rotate and zoom (virtually) on the reconstructed 3D model to view the anatomy from various viewpoints, a capability which is not possible with current intra-operative imaging techniques.

FIG. 7 depicts a representative screen capture of an exemplary interface including an additional Side View of the colorized 3D point cloud information of the cylinder portrayed on FIG. 6. Using the SSL thumbstick (FIG. 5), the surgeon is able to move, rotate and zoom (virtually) on our reconstructed 3D model without the need to reposition the laparoscopic camera or take her/his hands of the laparoscopic tools.

FIG. 8 depicts another representative screen capture of the 3D viewer from our the interface showing a heart model reconstruction from a different viewpoint, further displaying the capabilities of novel viewpoints of the SSL system disclosed herein which are different from the 2D color camera's (fixed) viewpoint.

FIG. 9 is a depiction of exemplary results of the SSL system disclosed herein. The first column in each row shows the white light images of each experiment. The second column shows the associated blue pattern image which is used for the 3D reconstruction. The third column shows the resulting depth images after corresponding the pattern images with the template image, and the fourth column shows the colorized photo-realistic 3D reconstructions. The top-row shows results of the calibration cylinder, to gauge numerical accuracy of the SSL system. The second and third rows show results on plastic organs of a heart and a brain segment, respectively. In each example, the 3D structure of the objects are accurately captured and the SSL system allows for rotating and zoom-in views on the reconstructions intra-operatively.

### DETAILED DESCRIPTION

Structured light projects a known pattern of light into a scene and measures this pattern with a camera. By knowing the structure of the pattern of light received by the camera, it is possible to compute 3D information. However, implementing this in-vivo during laparoscopy is difficult. The appearance of the surgical site must be maintained while implementing a Surgical Structured Light (SSL) system. Projecting a pattern of visible light onto the surgical site during laparoscopy, which is allowed to be perceived by the surgeon, could undesirably alter the surgeon's interpretation of the image and confuse or distract the surgeon. By analyzing how the light pattern is distorted by the 3D environment, structure can be accurately deduced in real-time using computational techniques.

The present subject matter provides for a functional SSL for use during laparoscopy, that maintains the view of the surgical site without distractions. In one exemplary embodiment, and in order to minimize distraction, a frequency of light is used that is not perceived by the human eye, such as infrared (IR) light. Using this method, dense 3D information is captured from a “single shot”, meaning only one image of the projected pattern is necessary to estimate 3D measurements. In one embodiment, an IR laser light source is diffracted with a special grating with a known pattern and an IR camera images the scene with the projected pattern and allows for a processor within the user interface to compute a depth image for presentation to the surgeon. In this exemplary embodiment, because the pattern is in the IR wavelength, the human eye cannot see the pattern and the view of the scene containing the projected pattern with a white light camera is uninterrupted. Additionally, an RGB color can be assigned to each 3D point in order to recover a photorealistic 3D reconstruction in real-time.

This capability allows for registration of pre- and intra- operative imaging, online metric measurements of tissue, and improved navigation and safety within the surgical field. By adding this 3D component minimally-invasive surgery would be easier to learn and more widely used.

The present disclosure provides a laparoscope with twin parallel optical channels to both transmit and receive light information to and from the scene and allow for triangulation. A beam splitter is placed at the tip of the device. One optical channel projects an IR pattern which is obtained from a laser point source dispersed from a special optical grating. The other optical channel receives all light from the scene, and a bandpass filter sends the white-light to a standard color camera and the IR light to an IR camera. The white light camera displays the “normal” image to the surgeon, while the IR camera uses the projected IR pattern to reconstruct the scene in 3D, in real-time. Further, real-time texture mapping of the geometry using the 2D viewing textures can be performed since the images are already registered (i.e. the system can identify which particular pixel(s) imaged a 3D location, thereby allowing for the 3D image to be combined with the 2D image).

In some embodiments, each channel of the two-channel laparoscope comprises an ocular lens adapted for the attachment of the camera and projector. White light is delivered through the ring of optical fibers around the periphery of the laparoscope. An independent housing is provided for the ocular lens of each channel, configured to enable coupling of a new headpiece to both ocular lens housings.

In another exemplary embodiment, the system can employ high-frequency white light “bursts” which would not be seen by the human eye, but could still be imaged by fast cameras, and would not distract the surgeon. The frequency of such bursts can be varied, as so desired, and is bounded only by the specifications of the particular camera model chosen for a given application.

In yet another exemplary embodiment, the system can employ a predetermined band of visible light to create the projected pattern instead of the invisible light embodiment (e.g. IR) as described above. In this configuration a notch filter is included which serves to remove this patterned band of visible light from the physician's view.

The imaging systems and techniques disclosed herein meet the need for 3D imaging for minimally invasive complex intra-abdominal and intrathoracic operations and significantly improve the experience of the surgeon and potentially improve outcomes for patients. This disclosure allows for live, dynamic 3D measurement capabilities of a surgical scene in real-time. It has been proven to work better than stereo imaging processing and has a large host of applications. This technology can be used for surgery, but can additionally be made smaller to work in other parts of the body, with or without a laparoscope, and applied to different types of medical procedures where 3D information “on-the fly” is helpful.

The disclosed subject matter provides a new approach to manufacturing and designing endoscopes that overcomes current limitations of visualization techniques used in minimally invasive surgeries by employing a system that visualizes internal body parts in three dimensions. A pattern of structured light, either in the infrared spectrum or a reduced band of visible light in conjunction with a notch filter, is projected on the tissue of interest by the endoscope. The pattern is detected by the accompanying camera system and subsequently converted to three-dimensional images that can be viewed by the surgeon. This technology can be used for visualizing internal structures in all areas of medicine and beyond, where depth perception can be a critical parameter, including in all minimally invasive surgeries, forensic applications, military applications, and law enforcement.

In one embodiment, the Surgical Structured Light system of the present disclosure may be constructed using a DLP Pico Projector (Texas Instruments/DigiKey), coupled with an endoscope for insertion. In other embodiments, a small diameter flash light is used. In some embodiments, the flashlight includes a cone at the end to make it into a point source. In some embodiments, a transparency is provided with a pattern to project. In some embodiments, the pattern is placed at the end of an endoscope. In some embodiments, a pattern is projected and an high-dynamic range (HDR) camera is used to view it so that the pattern is visible to the HDR sensor for reconstruction, but can be easily filtered out to a lower bit-depth image for viewing by the surgeon so the surgeon's view is not obstructed by the pattern. In some embodiments, a camera and projector combination is used that both images the scene and projects a pattern at a frequency that is not detectable to the human eye, again so the surgeon surgeon's view is not obstructed by the pattern.

Additional embodiments of the present subject matter use a genetic algorithm to evolve the best pattern to project. In some embodiments, the genetic algorithm uses an HDR grayscale camera to: take the desired image without any projected patterns; design the ideal pattern (high frequency random noise, vertical stripes, etc); start out half-way between the ideal image and the ideal pattern; evolve new patterns (pixel values can range b/w 0-255);

the fitness function simply deconvolves and then creates an 8-bit image and weights error from the ideal image after deconvolution along with how close the evolved pattern is to the ideal pattern. The final result will image a projected pattern on a scene in HDR format, deconvolve with the pattern, reduce to 8-bit format, and be able to perform structured light on the HDR image, all while retaining the ability to show the filtered image without the pattern included therein.

Laparoscopy, part of the larger class of surgical procedures known as endoscopy, is minimally invasive surgery through small incisions on the abdomen. It is often cheaper and less stressful for minor surgeries than laparotomies, which use significantly larger incisions. Typical procedures entail surgical tools, an imaging device, and a light source being inserted through the small incisions into a working space created by pressurizing the abdomen with CO₂.

Laparoscopy is widely used to conduct surgeries for hernia, abnormal growth removal, biopsy, organ removal (gallbladder, appendix, ovaries, etc.), etc. as well as diagnostic procedures to check for infertility, abnormal growths, chronic pelvic pain, chronic pelvic inflammatory disease, endometriosis, ectopic pregnancy, tube/ovary torsion, etc.

Standard laparoscopes, either (1) telescopic rod lens systems connected to a video camera or (2) digital systems with lens replaced with a charge-coupled device, generally provide 2D imagining of the surgical site. Inherently, these images lack depth perception information and can slow surgical performance.

Conceptually, the structured light techniques disclosed herein project a known pattern of light into a scene and measure the pattern with a camera. 3D information can be computed by looking at the structural changes in the light seen by the camera.

In accordance with an aspect of the disclosure, the Surgical Structure Light (SSL) system disclosed herein adds 3D imaging capability to laparoscopic procedures by adding a structured light source and detector to the standard laparoscope.

In an exemplary embodiment, infrared (IR) light is chosen, for the SSL platform because patterned visible light could interfere with the surgeon's view of the surgery site on the 2D image. A laparoscope with twin parallel optical channels provides light (visible and structured IR) transmission and reception to and from the surgical site while allowing for triangulation. The IR light is delivered via a dispersion unit containing a special optical grating to produce the pattern to which the IR camera compares the captured IR light in order to generate the 3D image.

All light received back from the surgical site (i.e., visible light, and invisible structured IR light) is split by a band pass filter to its respective visible or IR camera. The IR system color-codes its output (to denote depth) in real-time to display a 3D rendition of the surgical scene alongside the standard 2D image. Furthermore, real time texture can be added to the 2D image since the 3D images are already registered.

Additional embodiments of the present subject matter use high-frequency white light “bursts” instead of IR light for 3D imaging. The bursts are not be detectable by the human eye, i.e. no surgeon distraction, but can be imaged by high-speed cameras. Additionally or alternatively, the system can employ a predetermined band of visible light to create the projected pattern and a notch filter which serves to remove this patterned band of visible light from the physician's view.

The present subject matter has many applications including: Laparoscope adaptation for laparoscopic procedures; endoscope adaptation for 3D imaging during other endoscopic procedures; when configured on a smaller scale (e.g. no laparoscope) the SSL system can be used in medical monitoring applications where real time 3D imaging is helpful; 3D sensing for other applications such as video gaming systems, robotics, filming, interactive control, vehicle sensors, etc.

The present subject matter leads to better surgical procedures by enabling the following: faster operations; prevention of inadvertent injuries of structures adjacent to the surgical site; precise registration of intra-operative images with preoperative images; intra-operative planning; and real-time, accurate intraoperative measurements.

In one embodiment, the improved 3D laparoscope of the present disclosure comprises a lens splitter, an IR light source (e.g. laser or LED) with a dispersion unit (e.g. grating for laser, or mask for LED) and an imaging head. The system of the present disclosure may project any of several patterns that maximize information content/retrieval.

In other embodiments, the system of the present disclosure can use any of several wavelengths of light to create the projected pattern (including ultra-high speed white light projections, as well as a band of visible light in conjunction with a notch filter) that are not perceived by the surgeon's eye.

One embodiment of the present disclosure comprises software including registration and modeling software to take the 3D point clouds, build meshes from these clouds, and texture map the 2D imagery onto the models. This software is operated by processors housed within the user interface which includes a display for the surgeon's viewing of the surgical site.

The system of the present disclosure may be used in in-vivo animal environments and provides optimal: 1) accuracy of reconstructions, 2) frame rate for reconstruction of models, and 3) interface with the surgeon. The present disclosure is useful to healthcare providing institutions (hospitals, specialty surgical centers, etc.) providing endoscopic treatments. It is also useful in other fields including surveillance equipment (forensic, law enforcement, and military applications) and manufacturing applications.

The present disclosure provides for real-time, dynamic 3D visual information of the surgical environment using a standard laparoscopic setup. Although current laparoscope technology can provide perceptual depth information, these technologies require the surgeon to view stereo cameras and mentally compile a variety of images together so as to fuse a complex virtual image in his/her brain in order to perceive depth information. Often this is a time consuming and error prone endeavor which can distract the surgeon from safely operating the surgical tools, as well as lead to undue surgeon fatigue.

Prior to the present disclosure no methods existed to computationally provide this information for the purposes of measurements and automation. Attempts had been made to use stereo vision with computer vision techniques to estimate dense 3D information, however the inevitable existence of texture-less areas and significant lighting challenges make this an incredibly difficult, if not impossible, endeavor to perform robustly in a commercial environment. The system disclosed herein overcomes these drawbacks and provides for a 3D image generation with greater spatial density, and thus more accurate modeling of the surgical site.

The present disclosure solves the aforementioned shortcomings of conventional laparoscopic imaging systems by using a technique called Structured Light (SL), whereby a known pattern of light is projected into the scene and then imaged with a camera system. By analyzing how the light pattern is distorted by the 3D environment, anatomical structure can be accurately deduced in a real-time sense using computational techniques. One concern may be that the projected light could potentially distract the surgeon by disturbing the visual scene, and this has been the approach taken in the past. The present subject matter alleviates this by projecting light which is not perceived by the surgeon (e.g. in the infrared (IR) wavelength; high frequency white light bursts; or band of visible light coupled with a notch filter) thereby making the projected information invisible to the human eye. There is precedence for using IR light in-vivo, however these procedures were directed towards fluorescence techniques for visualizing tumors, and did not provide the structured light reconstruction of 3D images, as disclosed herein.

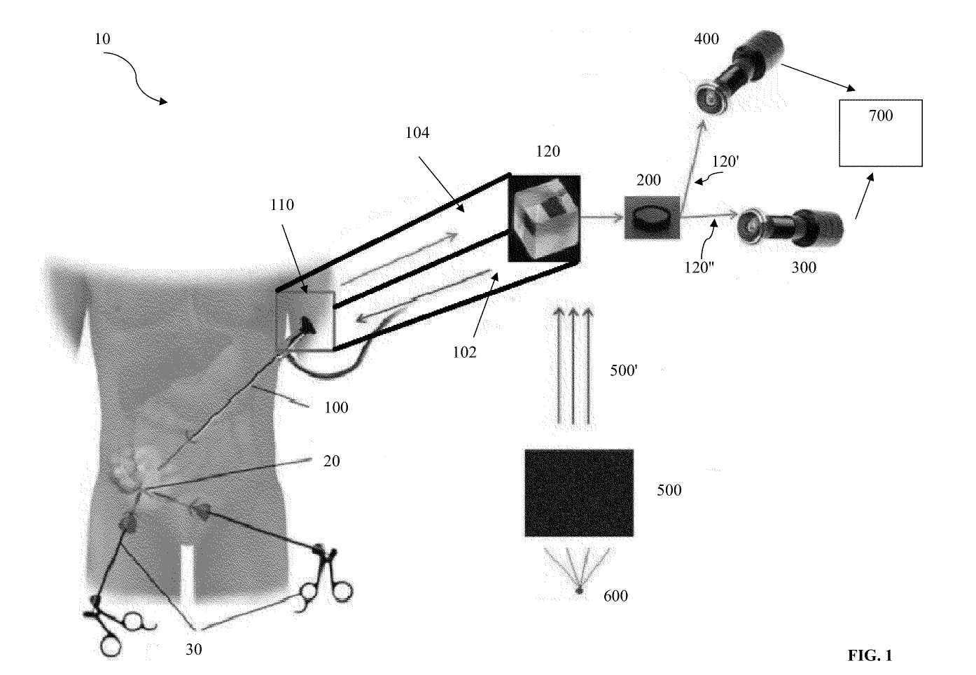

One embodiment of the present disclosure includes a hardware setup that begins with a dual, parallel optical channel laparoscope. Some existing laparoscopes use this dual channel technology for perceptual stereo, however the device disclosed herein includes a removable head at the imaging end which allows for 3D reconstruction and metrology functions to these existing dual channel laparoscopes. The system 10 of the present disclosure, as shown in FIG. 1, provides a removable head piece 110 which takes the place of the cameras at the tip of conventional dual channel laparoscopes. In the exemplary embodiment illustrated, the removable head piece 110 is positioned external to the body. A light source 600 is provided for generating light at a non-visible frequency (e.g., IR light) which is passed through a dispersion unit 500 that is configured with a predetermined structure to diffract the IR light into a known pattern 500′. In some embodiments the light source 600 can be a laser and the corresponding dispersion unit 500 includes a dispersion grating. In other embodiment the light source can be a light emitting diode (LED) and the corresponding dispersion unit is configured as a dispersion mask. A first channel 102 of the laparoscope is configured to project this non-visible patterned light into the body and thus serves as a Projection Channel. A second channel 104 is configured for imaging light from the body and thus serves as an Imaging Channel. Using the Projection Channel 102, a predetermined IR light pattern is projected through the laparoscope into a target area at the surgical scene 20. In addition to this projected IR light, the visible light (the source of which can be ambient operating room lighting and/or a directed beam, e.g., flashlight) for inspection by the physician is captured by the system disclosed herein. Therefore, the system and methods disclosed herein employs a first camera 300 for capturing the patterned invisible or undetected light, and second camera 400 for capturing the visible light (e.g., IR and white light color cameras, respectively). These cameras are in communication with the user interface 700 which houses a processor(s) for computing and displaying 3D images and measurements to the surgeon.

To facilitate the precise placement of both cameras compactly, a beam splitter 120 is positioned at the tip of the laparoscope 110. A beam splitter 120 is an optical device which is capable of splitting a beam of light into a plurality of discrete rays. Therefore, all of the light which exits the body through the Imaging Channel 104 first goes through the beam splitter 120. The beam splitter 120 splits the light into two separate beams. One beam 120′ goes to a standard, high-definition white light camera 400 which is used for normal viewing by the surgeon. The white light camera 400 is not sensitive to IR light and so the projected pattern remains invisible to the surgeon viewing the procedure via the images presented by the white light camera 400. The other beam 120″ is passed through an IR band-pass filter 200 which is designed to filter out all visible wavelengths of light and only let pass-through non-visible IR light. This causes only the patterned IR light to pass to the IR camera 300, which is then able to view the distorted view of the IR pattern which was passed through the Projection Channel 102.

In another exemplary embodiment, the IR light source 600 of FIG. 1 is substituted with a light source which produces a ray of visible light. Additionally, the band pass filter 200 is substituted with a notch filter. Notch filters, also commonly referred to as band-stop or band-rejection filters, can transmit most wavelengths with little intensity loss while attenuating light within a specific wavelength range (the stop band) to a very low level. Accordingly, notch filters are effectively the inverse of bandpass filters, which offer high in-band transmission and high out-of-band rejection so as to only transmit light within a small wavelength range. The notch filter creates a restricted region over a given range wavelengths. In an exemplary embodiment, the notch filter of the presently disclosed SSL system is configured to restrict wavelengths within the blue spectrum, however alternative portions of the spectrum can be restricted if so desired. In some embodiments the notch filter is positioned within the beam splitter 120 wherein the light is both split and filtered simultaneously. In other embodiments, the notch filter is disposed between the beam splitter 120 and the camera 300. The use of a visible light source in combination with a notch filter is advantageous in that it affords the same quality and resolution of imagining, while alleviating the need for specialized, and costly, equipment such as an IR camera.

By knowing the positions and orientations of all cameras and light projectors, it is possible to reconstruct the 3D information in the scene densely using triangulation techniques in computer software. In addition, because there is a white light color camera 400, it is also possible to assign RGB color information to each 3D scene point, thereby producing a photo-realistic 3D reconstruction. Additionally, the texture of the target site 20 can be automatically registered with the depth data since the same optical channel 104 is used for both depth and texture. Accordingly, the system disclosed herein provides a Surgical Structured Light (SSL).

In accordance with another aspect of the disclosed subject matter, a processor executing software can be provided which encompasses both the algorithms required to reconstruct the scene with the observed imagery as well as the user interface (UI) display 700 which is presented to the surgeon. The UI is such that the introduction of 3D information can be seamlessly blended into the surgeon's procedural routines without effort. In one embodiment, 2D imagery is combined with the dense 3D information, which can take the form of a side-by-side view of 2D and 3D imagery. Additionally, a button (not shown) on the laparoscope 110 which can switch between the views on-demand, or a blending of the 2D video onto the 3D structure to view both images simultaneously, if so desired. As would be understood by one of ordinary skill, the UI design will differ depending on the particular application of the disclosed structured light system (e.g., assisted navigation may require a different presentation than a virtual ruler).

There are various uses of such a 3D imaging system. Although the disclosed SSL system is not a perceptual system, it is possible to provide real-time 3D measurements of individual points in the scene 20. This can be useful during a procedure when the surgeon needs to know (quickly) the precise distance between two locations in the body. Therefore, the SSL would be a seamless means of performing such a measurement because the software has 3D locations of all visible points in the scene. As such, SSL effectively provides a virtual ruler without the injection of any physical tools into the body. This could be especially useful if the surgeon is performing a biopsy at a location pre-determined from pre-operative imaging techniques to a location which is known metrically, but isn't visibly obvious in the image.

Another useful application is in registering the intra-operative 3D video available via the SSL system to pre-operative images. This is a powerful tool for the surgeon as she tries to identify structure in-vivo. It requires accurate geometry, and the SSL system provides dense 3D information from which this can be extracted. Because it is possible to compute photo-realistic 3D information, one could also potentially provide novel viewpoints during fine-scaled manipulations, for example, in the case where a side-view is more useful in navigating a tool to a site, and this is currently impossible since all views must come from the point-of-view of the camera. Another possible use is to perform very accurate small surface reconstructions of anatomy. Lesions, tumors and other surface anomalies may not be identifiable with the human eye, and the reconstructions provided for via the SSL system may be able to recover very dense surface models that can be used to identify abnormalities in-vivo.

There are numerous additional applications for this type of real-time dense 3D information is obtainable. Surgical robotics is an obvious domain for application of the SSL system, whereby more automation may be possible with the existence of better 3D measurement capabilities. The present subject matter overcomes the limitations currently enforced by using 2D information from cameras.

In some embodiments, the system of the present disclosure integrates the software for 3D reconstruction and User Interface with the device. The system of the present disclosure can be used to perform surgical mockup experiments to evaluate the system in advance of animal and human use.

The present disclosure includes design of optical system to allow dense 3D reconstruction, and an intuitive and simple user interface to allow the surgeon to access the 3D data as needed. The system of the present disclosure may be combined with a surgeon education program educating surgeons on the new technology.

Exemplary Hardware

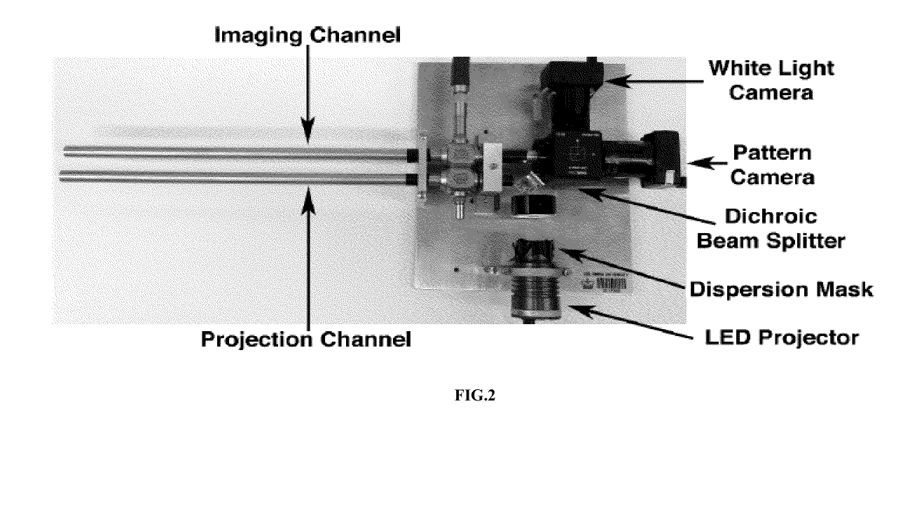

For purpose of illustration and not limitation, FIG. 2 depicts an exemplary embodiment of the SSL system including 10 mm laparoscopes for the projection and imaging channels. A small-band Blue LED projector with a GOBO mask projects the pattern of light into the scene while the dichroic beam splitter relays the blue light to the pattern camera and all other light to the white light camera. By adapting the SSL system to standard laparoscopes and ensuring that all customized hardware is outside the patient's body, adoption of this technology into existing operating rooms is significantly simplified.

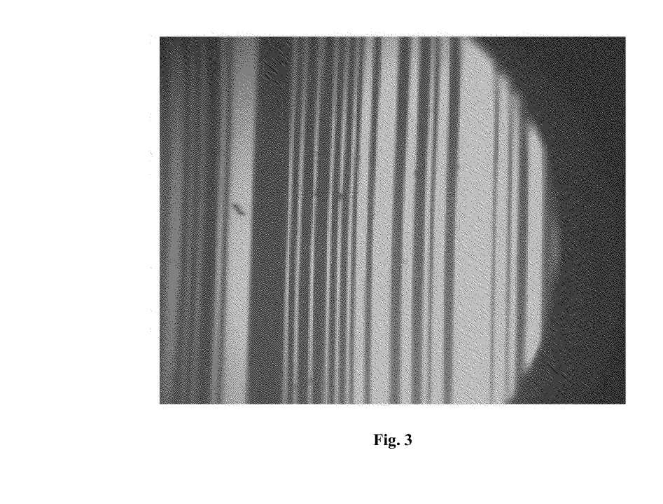

The SSL system depicted in FIG. 2 employs two standard, side-by-side 10 mm laparoscopes, two Point Grey FireflyMV color cameras, and a small-band 460 nm blue LED light projector coupled with a custom-designed GOBO pattern dispersion mask. The GOBO mask is attached to the end of the LED light projector and projected down one of the laparoscopes (the “projection channel”). The smallband light projector is chosen in a range of the visible spectrum that can safely be removed without the surgeon realizing a strong difference in the color of the images when presented with this small band removed via optical filters. The exemplary custom mask (see FIG. 3) is designed as a barcode pattern of vertical lines (though other patterns are within the scope of the disclosed subject matter). Because the exact range of blue light contained within the pattern is known, it is possible to attach a centerpass optical filter on a camera coincident with the second laparoscope (the “reception channel”) so that the camera sees nothing but the blue pattern light for the purposes of structured light reconstruction. This simplifies the process of identifying the pattern in the surgical scene amongst all other light and structures that are present. A standard white light source is simultaneously delivered through a ring of optical fibers around the periphery of the “reception” laparoscope, exactly as all standard laparoscopes deliver white light to the surgical field. This white light also has the small-band blue wavelength removed to avoid confusion with the blue light being delivered by the pattern projector.

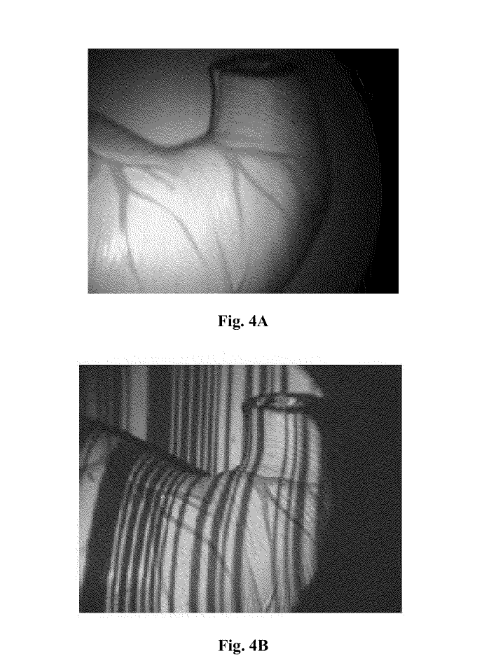

The reception channel receives all of the light in the scene, both the blue pattern light and all of the standard light of the surgical site. At the proximal side of the laparoscope (outside the patient's body), a dichroic beam splitter splits the outgoing light into two orthogonal directions. The dichroic beam splitter also filters the light so that in one direction, only the blue light which was delivered through the LED pattern projector is relayed, while the orthogonal direction receives all other light (minus the blue pattern light). FIG. 4A shows a plastic organ model (of a stomach) and the associated projected mask pattern with the blue light is shown in FIG. 4B. In this way a “pattern camera” and an “imaging camera” are provided. Because the pattern camera only receives the blue pattern light, the detection and decoding of the pattern becomes less complicated than other structured light setups. Additionally, because only using blue light for this camera (in this exemplary embodiment), the Bayer pattern is removed (on the pattern camera only) so that every sensor unit receives unfiltered blue light. Finally, the imaging camera has the narrow-band blue pattern removed (by the dichroic beam splitter) and so the surgeon is not distracted by the blue barcode during the procedure, and the 3D reconstruction becomes “invisible” to the surgeon.

Exemplary Software

Single-shot structured light uses a single image as a template of the known pattern and matches subsequent images with 3D structure to the single image and decodes the warp of the pattern due to the 3D structure to recover the 3D information in the scene. Additionally, a procedure is performed whereby an image of the barcode template is taken at a flat, parallel plane at a known distance from the tip of the laparopscope distal tips. The settings of the cameras are setup to maximize the contrast of the edges in the vertical line barcode pattern. Then, for subsequent frames, the warped barcode pattern is detected and matched, line-by-line, with the correspondences to the template pattern on each scanline of the image separately. In this way, as the 3D structure of the scene warps the template, the horizontal disparity changes according to the amount of 3D structure compared to the flat image of the same template image.

To demonstrate the accuracy of the system, each image pair is labeled to identify all of the vertical lines in the image from the pattern. Next, each scanline is analyzed one-at-atime, and match-up scanlines between the test and template images. As each “bar” in the template pattern begins, the corresponding bar endpoint is automatically found in the test image, assuming that horizontal scanlines correspond due to stereo epipolar undistortion. In order to undistort the images from lens distortion, both the pattern and imaging camera are pre-calibrated using standard camera calibration techniques. Also, because there are two cameras, the pattern and imaging cameras are calibrated to each other as a stereo camera rig to recover the stereo extrinsics as a rigid body rotation and translation, allowing one to lookup RGB color values for each 3D point recovered.

Because one can only correspond the edges of a bar in the pattern, one can linearly interpolate the horizontal disparity in between every pair of lines representing a complete “bar” in the pattern. This assumption requires a smooth transition of depth within the confines of a single bar, and by using thin enough bars, this can be reasonably achieved. In the end, a full horizontal disparity image between a test image and the template image can be computed.

1) Depth From Disparity: In a typical stereo setup, depth can be recovered from horizontal disparity using the following relationship:

$$z = \frac{b * f}{d} \qquad ( 1 )$$

In this equation, z is the depth (in meters), b is the calibrated horizontal baseline between the cameras (in meters), f is the (common) focal length of the cameras (in pixels), and d is the disparity (in pixels). Based on this relationship, as disparity approaches zero, the depth approaches infinity. However, in the exemplary customized structured light setup, because an image of the template is captured at a known (positive) distance from the cameras, zero disparity now corresponds to this known distance rather than infinity, and Eq. 1 must be adjusted:

$$z = \frac{b * f}{{d}_{off} - d} \qquad ( 2 )$$

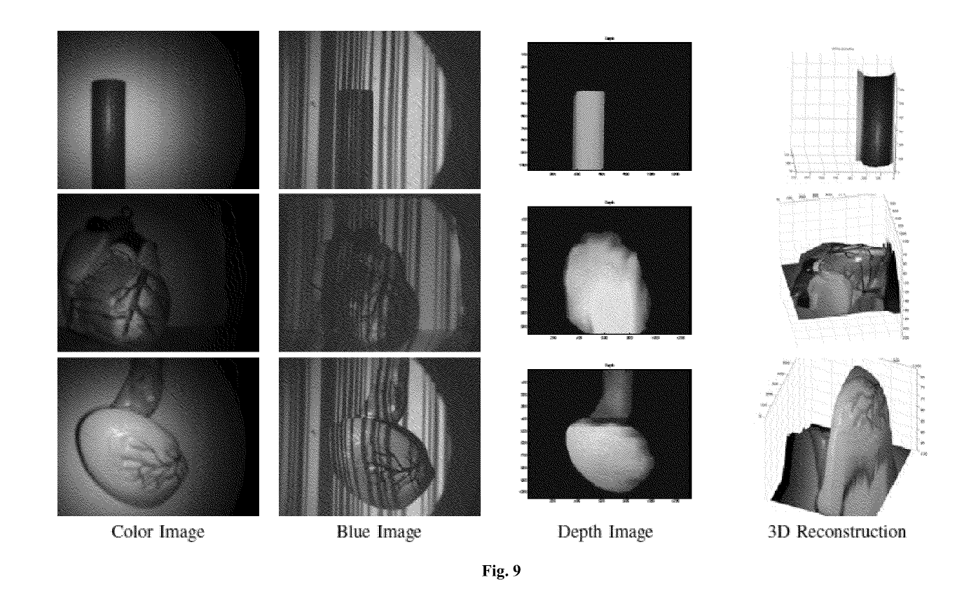

In this equation, d_{off} represents the disparity offset due to the distance to the known template image in order to renormalize zero disparity to the correct depth. The value of d_{off} is computed empirically using a calibration cylinder object (FIG. 9, top-row) by mapping known depth values to known disparity values and recovering the best fit estimate of d_{off}. Once a depth value at every pixel is known (and the focal length (fₓ, f_{y}) and principal point (cₓ, c_{y}) of the pattern camera), a 3D point at every pixel can be recovered as:

$$x = z * \frac{u - {c}_{x}}{{f}_{x}} \\ y = z * \frac{v - {c}_{y}}{{f}_{y}} \qquad ( 3 )$$

where (u, v) is the pixel corresponding to the depth value z. In this way, given an arbitrary image, it is possible to:
Compute line-by-line correspondences of the vertical lines in the barcode from a test image to the template image
Linearly-interpolate the horizontal disparity in-between every line pair correspondence
Convert disparity to depth using Eqn. 2
Recover the 3D point cloud using the depth image at every pixel using Eqn. 3

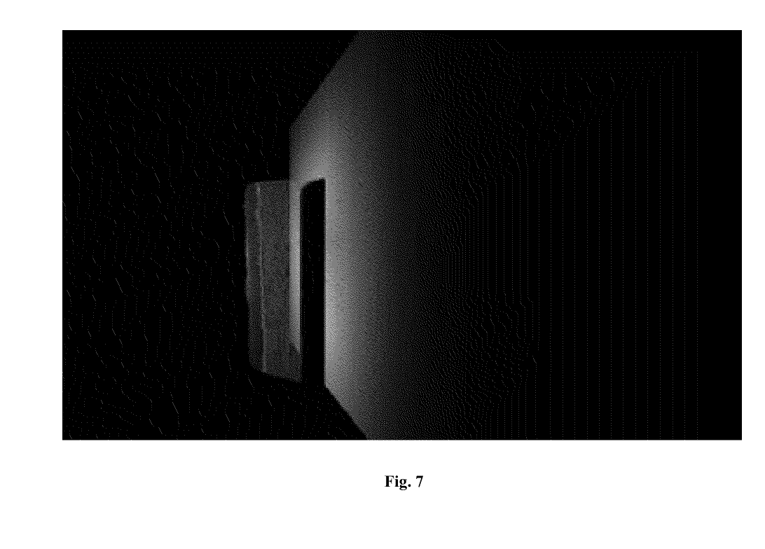

2) Colorizing the Point Cloud: Finally, because it is desirable to display a graphically-pleasing colored 3D point cloud (FIG. 7), the stereo extrinsics is used between the pattern and imaging cameras to assign an RGB color to every 3D point recovered from the depth image. To do this, first compute every 3D point with respect to the blue camera, as described above. Next, apply the rigid-body stereo extrinsics recovered from the camera calibration to represent this 3D point with respect to the imaging camera. Finally, apply the imaging camera's intrinsics to look-up the corresponding RGB color information for this 3D point, allowing the system to render a photo-realistic 3D rendering of the scene on-the-fly.

3) Developing a User Interface: To allow the surgeon to visualize and perform online transformations on the 3D reconstructed images during an in-vivo procedure, a representative user interface is provided with the SSL system disclosed herein.

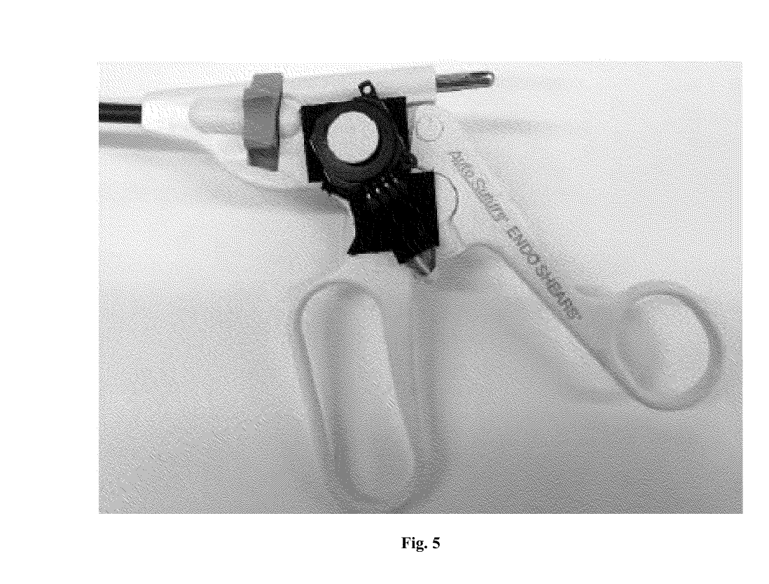

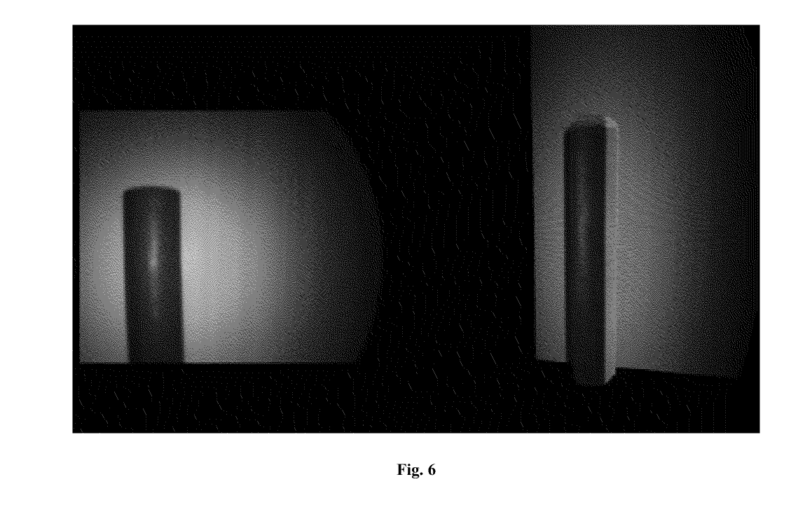

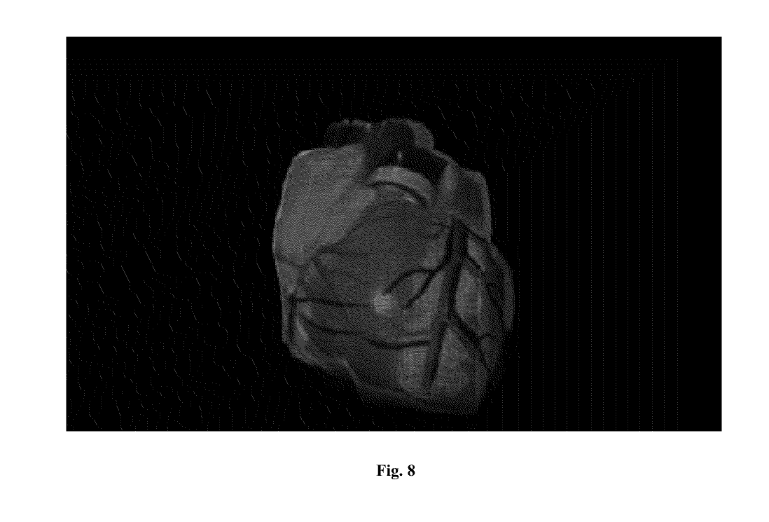

Using the exemplary SSL Thumbstick shown in FIG. 5, a 2-axis button joystick laparoscopic, the surgeon is able to operate in three different modes: 1) Standard live 2D image mode, 2) Side-by-side mode (FIG. 6) of the live 2D image and the 3D reconstructed view, and 3) 3D reconstructed view mode (FIGS. 7-8).

FIG. 6 shows the side-by-side mode of a cylinder where the surgeon can see both the live 2D image from the online in-vivo procedure and the 3D reconstructed view, allowing her/him to manipulate the 3D display, for example, to zoom in on an organ or rotate the viewpoint while maintaining full view and locus with the 2D image on the other side.

FIGS. 7-8 show the 3D reconstruction view mode of a cylinder and a plastic organ (Heart). Since a 3D model has been reconstructed, the surgeon can rotate and translate the model to obtain novel views of the anatomy that are not possible with a standard 2D image.

Accordingly, the approach provided herein is unobtrusive to the surgeon and complies with Schneiderman's Visual Information-Seeking Mantra, providing the surgeon the amount of information s/he needs exactly when s/he needs it. In addition to that, the surgeon is not required to take her/his hands off the laparoscopic tools at any time or move back and forth the actual laparoscopic camera to obtain a different view. Such capabilities are not possible with current intra-operative imaging techniques.

The accuracy of the SSL system disclosed herein is demonstrated using ex-vivo data on both a cylinder calibration object (FIG. 9, top-row) as well as various plastic organs (FIG. 9, rows 2-4). In order to reconstruct any object, a template image must first be captured, showing an unobstructed view of our barcode pattern on a flat parallel plane. An example of this is shown in FIG. 3. Additionally, one must estimate the d_{off} parameter described in Eqn. 2 which maps zero-disparity values to a known positive depth distance. This is done empirically using the cylinder object to estimate d_{off} by corresponding disparity values to known depth values.

Next, an image of the calibration cylinder is captured, with a pre-measured diameter of 17.50 mm. The first row of FIG. 9 shows the white light and blue pattern images in the first and second columns, respectively. Next, the pattern lines in the cylinder's blue pattern image are matched to our template pattern image, recovering the horizontal pixel disparity at every pixel in the image, and then recover the depth image using Eqn. 2 as shown in the third column. Finally, using Eqn. 3 the cylinder object is reconstructed in 3D and assigned an RGB color to each 3D point using the technique described above in the “Colorizing the Point Cloud” description, producing a dense, photo-realistic 3D reconstruction of the cylinder in the fourth column of FIG. 9. To measure the accuracy, the diameter of our cylinder reconstruction is calculated, resulting in an error of 0.20 mm. Furthermore, the distance from the tip of the laparoscope to a flat plane at a known distance immediately anterior to the object was analyzed, positioned at exactly 100.00 mm (measured with calipers) along the optical axis. The reconstruction showed an error of 0.30 mm to this flat plane. It is noteworthy that typical errors in stereo reconstruction fall mostly along the optical z-axis of camera systems, and so the sub-millimeter error rate along this axis direction demonstrates the effectiveness of the SSL system disclosed herein.

To further test the SSL system, reconstructions on 3 plastic organs, shown in FIG. 9 on rows 2 (Heart), and 3 (Brain Segment) were performed. For each example, the first column shows the white light image, noting that the blue pattern is completely removed through the dichroic beam splitter so that the surgeon's view is unobstructed by the barcode pattern. The second column for each shows the blue pattern camera with the object that is desired to be reconstructed. The third column shows the resulting depth image after matching the pattern in the blue camera to the pre-captured template image, and finally a colored, photo-realistic 3D reconstruction in the fourth column.

One advantage of the SSL system disclosed herein is the ability to assign realistic color information to each 3D point in the reconstruction. The SSL system disclosed herein achieves this solely due to the fact that the white light color image has the pattern removed, whereas in other implementations of MIS structured light the projected light source would show up in the reconstruction, and so no color information can be provided. This allows the surgeon to approach the object (virtually) from different angles and viewpoints through the user interface, and the color allows them to make sense of the information much easier than an uncolored reconstruction might.

The particular hardware and software described above are presented for purpose of illustration and not limitation. For example, the present disclosure includes using more sensitive cameras with larger image sensors as well as experimenting with other types of laparoscopes and LED light sources and wavelengths to improve or enhance the real time speed of the SSL system disclosed herein

Similarly, the exemplary pattern for the GOBO LED mask discussed above was a barcode of vertical lines. Laparoscopes are designed to magnify the scene quite a bit at small distances to optimize for typical surgical scenarios. A quick inspection of the exemplary pattern images yields a relatively coarse pattern with respect to the overall scene. This can be overcome by interpolating the horizontal disparity in between every pair of lines representing a single bar in the pattern. However, if a bar is too large then this interpolation may overly smooth the object at some locations and lose important detail that the surgeon may wish to capture. Accordingly, the SSL system disclosed herein can employ a more dense pattern of vertical lines so that so that the interpolation strategy is performed on smaller lines, resulting in less loss of detail. Additionally or alternatively, a pattern of random dots with a block matching scheme can be employed. By achieving a denser pattern, the SSL system will be more robust to extreme changes in the 3D structure of an object, because the more dense the pattern, the less individual locations in the pattern can move due to 3D surface geometry. In this way, the decoding strategy will simplify and become more robust and accurate.

In accordance with an aspect of the disclosed subject matter, an important side effect of generating an accurate 3D model is the ability to do metrology on the organ model.

This will allow the surgeon to take online measurements in-vivo during a procedure that will assist her or him. This creates a virtual ruler and eliminates the need to insert a physical ruler into the body. Also the ability to manipulate the generated model to provide novel views while still viewing the 2D image may prove important to the surgeon. Also, with an accurate 3D model, the SSL system disclosed herein has the capability to register preoperative images with online in-vivo anatomy.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. An imaging system comprising:
a first camera;
a second camera;
a light source, the light source producing light at a frequency invisible to a human eye;
a dispersion unit, the dispersion unit projecting a predetermined pattern of light from the invisible light source;
an instrument, the instrument projecting the predetermined pattern of invisible light onto a target area;
a band pass filter, the band pass filter directing visible light to the first camera and the predetermined pattern of invisible light to the second camera;
wherein the second camera images the target area and predetermined pattern of invisible light to compute a three-dimensional image.

2. The system of claim 1, further comprising a user interface, wherein the user interface configured to display two dimensional and three dimensional images.

3. The system of claim 1, wherein the light source produces infrared light.

4. The system of claim 1, wherein the light source produces a continuous ray of light.

5. The system of claim 1, wherein the three-dimensional image is computed from a single image of the target area.

6. The system of claim 1, wherein the second camera takes real time intra-operative images of the target area.

7. The system of claim 6, wherein the system compares pre-operative images with intra-operative images.

8. The system of claim 1, wherein a RGB color is assigned to select locations of the second camera image.

9. The system of claim 1, wherein a processor measures at least one length of the target area.

10. The system of claim 9, wherein the processor calculates a depth of the target area.

11. The system of claim 1, wherein instrument is a laparoscope.

12. The system of claim 11, wherein the laparoscope includes a beam splitter disposed at an end thereof

13. The system of claim 11, wherein the laparoscope includes first and second optical channels.

14. The system of claim 13, wherein the first optical channel projects the predetermined pattern of light onto the target area.

15. The system of claim 13, wherein the second optical channel receives all available light from the target area.

16. A method for creating a three dimensional image comprising:
providing a first camera;
providing a second camera;
generating a ray of light at a frequency invisible to a human eye;
dispersing the ray of invisible light through a dispersion unit;
projecting a predetermined pattern of invisible light onto a target area;
receiving the predetermined pattern of invisible light and visible light from the target area;
directing visible light to the first camera and the predetermined pattern of invisible light to the second camera;
imaging the target area and predetermined pattern of invisible light; and
computing a three-dimensional measurement.

17. The method of claim 16, wherein the ray of invisible light is infrared light.

18. The method of claim 16, wherein the second camera takes real time intra-operative images of the target area.

19. The method of claim 16, wherein a processor measures at least one length of the target area.

20. An imaging system comprising:
a first camera;
a second camera;
a light source, the light source producing light at a frequency visible to a human eye;
a dispersion unit, the dispersion unit projecting a predetermined pattern of light from the light source;
an instrument, the instrument projecting the predetermined pattern of light onto a target area;
a beam splitter, the beam splitter directing a first ray of light towards the first camera and a second ray of light towards the second camera;
a notch filter, the notch filter filtering the second ray of light to the predetermined pattern of light;
wherein the second camera images the target area and predetermined pattern of light to compute a three-dimensional image.
